(19) United States
(12) Patent Application Publication
(10) Pub. No.: US 2024/0299156 A1
(43) Pub. Date: Sep. 12, 2024

(54) A SYSTEM FOR CONNECTING A BIONIC ORGAN TO A VASCULAR GRAFT AND A METHOD OF CONNECTING A BIONIC ORGAN TO A VASCULAR GRAFT

(71) Applicant: POLBIONICA SP. Z O.O., Warszawa (PL)

(72) Inventors: Michal WSZOLA, Warszawa (PL); Marta KLAK, Pila (PL); Andrzej BERMAN, Warszawa (PL); Tomasz BRYNIARSKI, Nowy Targ (PL); Tomasz DOBRZANSKI, Warszawa (PL)

(73) Assignee: POLBIONICA SP. Z O.O., Warszawa (PL)

(21) Appl. No.: 18/571,778

(22) PCT Filed: Jun. 30, 2021
(86) PCT No.: PCT/PL2021/050050

§ 371 Date: Dec. 19, 2023

(51) Int. Cl. A61F 2/07

(52) U.S. Cl. CPC A61F 2/07; A61F 2220/0075

(57) ABSTRACT

The invention relates to a system for connecting a bionic organ to a vascular graft and a method for connecting a bionic organ to a vascular graft under ex vivo conditions. Once connected, the vascular graft is situated within the bionic organ, and the stent is situated internally to the vascular graft and the bionic organ.

### FIELD OF INVENTION

This invention relates to a system for connecting a bionic organ to a vascular graft and a method for connecting a bionic organ to a vascular graft under ex vivo conditions. Once connected, the vascular graft is situated within the bionic organ, and a stent is situated internally to the vascular graft and the bionic organ.

### PRIOR ART

Various types of stents are known from the prior art. Stents for medical applications are predominantly designed to dilate blood vessels following stenosis thereof. An example of such a stent is described in European patent EP1608299, which presents a self-expanding or balloon-expandable stent whose expansion within a blood vessel can occur simultaneously from both ends of the stent until the stent becomes fully expanded. The stent described herein is further provided with markers in the actuating part, which allow for the device to be located inside a human body. A tip of the stent was termed as non-damaging.

Another document regarding stents, European patent application EP3160400, presents a stent for use in a human body, comprising a flexible tip that facilitates insertion into a target site, loops in a connecting part to securely fix the stent and X-ray markers used to assess a position of the stent. The stent provides for a connection that allows for fluids to flow between a recipient body site into which the stent had been inserted and another end of the stent. Such stent is not self-expanding and is connected to a catheter in order to supply fluids to or drain them from the body. Its use in the human body is temporary.

European patent application EP2146674, in turn, describes a self-expanding stent that is inserted into a blood vessel using a special mandrel. Such a mandrel has a sharpened tip which, when the former is inserted into a target site, breaks open to allow the stent to exit through said tip. The mandrel is then withdrawn, and the stent is expanded and remains in the target site.

A similar solution is presented in another patent, JP4857125, wherein again a stent is delivered to a target site in a mandrel that is equipped with a longitudinally breaking tip. Once inserted, the tip breaks and provides sufficient lumen for the stent to pass through it. The stent then self-expands, which keeps it in the target site.

A possibility was also described in the prior art of connecting blood vessels together using stents, for example as indicated in U.S. Pat. No. 9,241,782. The document describes a use of a self-expanding or balloon-expandable stent for connecting two blood vessels together. Such a connection may further comprise using expanding rings and clamps at both ends of the stent designed to securely fix the vessels to the stent. The stent may be present on the outside of the vessels or on the inside of the vessels. Stent-vessel connection is carried out as follows (for a stent on the outside of the vessel): 1) the stent in a compressed state is placed between the vessels that are spaced apart, which the stent is intended to connect, and the stent is expanded so that its diameter is greater than that of the vessels; 2) the vessels are placed in the stent; 3) the stent is contracted so that its diameter is smaller than the diameter of the expanded stent but larger than the diameter of the stent the compressed state; 4) the stent may be secured onto the vessels using expanding rings or micro-clamping stents (placed inside the vessels) so that the vessels are clamped between the stent and the ring at both ends of the stent. A plurality of such rings or other corresponding fixtures (flanges, clamps, etc.) may be used, but not necessarily. If a stent is used on the inside of the vessels, assembly process is similar, with rings (clamps) placed on the outer sides of the vessels. A mixed configuration is also possible, where one end of the stent is situated in the first vessel and the other end of the stent surrounds the second vessel.

Another US patent, U.S. Pat. No. 6,336,937, describes a possibility of using a self-expanding stent to bypass an embolic fragment of a blood vessel. In such a bypass, two expandable stent tips are placed in the blood vessel upstream and downstream of a blockage site, respectively, and are connected with a flexible tube (e.g. made of polymer). The length of the tube is selected as needed so as to provide a by-pass. In order to place the device in a blood vessel, the latter is incised where appropriate, and the tip of the stent is inserted inside.

The tip will expand, which will keep it fixed inside the vessel. Through the tube, blood will flow to another end of the bypass connection, placed in a similar manner in the blood vessel at the site behind the blockage.

US patent application US20200360126A1 describes a self-expanding or balloon-expandable stent used at a junction site of two vessels to prevent vessel stenosis following anastomosis. In order to perform the anastomosis, the vessels are connected by means of a suture, clip or another element used to connect the vessels. In this solution, the stent supports the structure and ensures that the vessels are properly fixed in relation to each other. The stent further prevents restenosis of the blood vessels. The blood vessels to be connected may have different diameters, so that the stent to be inserted thereto also needs to have a different diameter after it expands at either end. A change in diameter may be gradual along a length of the stent or it may be abrupt at a particular point of the stent.

### OBJECT OF THE INVENTION

The object of the invention is to develop a method for reliably and permanently connecting a bionic organ and a vascular graft together outside a living organism under ex vivo conditions. Said object was achieved by employing a system with a design suitable for this use.

### DESCRIPTION OF THE INVENTION

The invention relates to a system for connecting a bionic organ to a vascular graft which comprises a self-expanding stent, a casing and a mandrel with a breakable tip, wherein, in an assembled state of the system, the stent in a compressed state is placed in the casing, which casing holds the stent in a compressed state until the stent is removed from the casing, and a mandrel is placed inside the stent for removing the stent from the casing, wherein, in the assembled state, the length of the casing and the mandrel is in the range of 20 to 40 cm, the length of the stent is in the range of 10 to 40 mm, and the diameter of the stent is selected such that, in the compressed state, the diameter of the stent is less than diameter of the vascular graft and, when expanded, the diameter of the stent is greater than or equal to diameter of the vascular port in the bionic organ.

Preferably, in the expanded state, the diameter of the stent ranges from 0.2 mm to 50 mm, preferably from 1 to 20 mm.

In the assembled state, the casing and mandrel may have a length of 30 cm, and the stent may have a length of 20 mm.

Preferably, the mandrel comprises a circumferential notch that allows the breakable end of the mandrel to break off.

The breakable tip of the mandrel may be conical with a rounded end.

Preferably, the stent comprises at least one fixing loop, preferably it comprises two fixing loops, one at each end of the stent.

Preferably, the casing comprises a depth indicator.

Optionally, the system comprises a set of at least two samplers that allows for selecting the diameter of the stent to match the diameter of the vascular port, wherein the samplers have diameters of different sizes ranging from 0.2 mm to 50 mm, preferably from 1 mm to 20 mm.

The invention also relates to a method for connecting a bionic organ to a vascular graft ex vivo, wherein the connection is achieved by means of a system of the invention, which method comprises the following steps:
a) Inserting the system in the assembled state into the vascular graft
b) Partially removing the stent from the casing and attaching the stent to the vascular graft, preferably by means of a loop
c) Breaking off the tip of the mandrel, preferably at a circumferentially notched point
d) Inserting the vascular graft with the attached stent in the casing into the vascular port of the bionic organ
e) Removing the stent from the casing by using the mandrel
f) Removing the casing and the mandrel from the vascular port of the bionic organ.

They following may be used as the vascular graft:
a decellularized or recellularized vessel of animal origin, or
a preserved vessel of animal origin, or
an autogenous vessel, preferably a saphenous vein, or
a vessel harvested from a deceased donor and preserved, or
a vessel harvested from a deceased donor and non-preserved, or
a vascular prosthesis, preferably made of EPTFE (expanded polytetrafluoroethylene).

Prior to implementing the step a), the vascular graft may be decellularized and then populated with endothelial cells.

The bionic organ may be made using 3D bioprinting technology.

Preferably, the insertion depth in step d) is regulated by means of a depth indicator and corresponds to the length of the stent to be used.

Preferably, in step d) the stent is inserted to a depth of 10 to 40 mm, more preferably 15 to 30 mm, and most preferably to a depth of 20 mm.

Most preferably, the sequence of steps a) to f) is performed for each vascular port of the bionic organ.

### BRIEF DESCRIPTION OF FIGURES

The object of the invention in the embodiment is shown in the drawing, where:

FIG. 1 shows a stent 2 in its compressed state prior to its insertion in a vascular graft 4, where: 1 is a mandrel of the stent, and 3 is a casing of the stent;

FIG. 2 shows the stent 2 after it is fixed in the vascular graft 4 and before it is installed in a vascular port 7 inside a bionic organ 8, where 1* is the mandrel after the tip has been broken off, 5 is a depth indicator, and 6 is a fixing loop;

FIG. 3 shows the stent 2 connected to the vascular graft 4 after it is installed in the vascular port 7;

FIG. 4 shows a set of samplers with different diameters for selecting the diameter of the stent 2 for the vascular graft 4;

FIG. 5 shows the bionic organ 8, an artificial pancreas, connected through its vascular port 7 to the vascular graft 4 by means of the stent 2, and a flow of contrast medium across the bionic organ 8.

For the purposes of the present invention, a vascular graft is defined as any blood vessel of animal origin, such as for example a blood vessel harvested from a pig, cow, sheep and other animals, or of human origin, previously harvested from the human body and not connected with the human or animal body as part of implementation of the method according to the invention.

Preferably, such a vessel is previously decellularized, i.e. it is stripped of cells and foreign DNA in order to eliminate a possibility of tissue incompatibility with a potential recipient. Even more preferably, following decellularization, such a vessel is then populated with recipient cells. Decellularization may be achieved using any method known in the art, such as for example a flow method or using a static system where the vessels are in a detergent solution in a vessel placed on a shaker.

The term “bionic organ” or “artificial organ” denotes an artificially obtained three-dimensional structure that imitates any organ, such as pancreas, lung, heart, liver, etc. Preferably, the bionic organ is obtained using 3D bioprinting. The bionic organ comprises a vascular system, ending with vascular ports.

The method according to the invention provides a fast, tight and permanent connection of the bionic organ and the vascular graft.

The vascular graft thus connected together with the bionic organ may then be connected to recipient's vascular system, such as by means of a vascular suture (indirect connection using a vascular graft).

The connection method according to the invention may also be used to directly connect the bionic organ to the recipient's vascular system by directly connecting the organ to the recipient's vessel using an in situ stent. More specifically, such a connection is performed by making an incision on the recipient's vessel upstream of the connection point with the bionic organ and inserting a stent thereto in order to expand it and to secure the connection. The following options are possible for connecting the bionic organ to the recipient's vascular system in situ:

isolated arterial and venous vessels of the recipient in situ-here, steps a) to c) of the method according to the invention are replaced as follows: terminal vessels of the recipient (cut at one end) (e.g. epigastric vein and artery, internal iliac vein and artery) isolated over a distance of at least 30 mm are temporarily closed, preferably with a vascular clamp, and an incision is made therein near to the closure site, so as to introduce the system according to the invention through the opening provided towards the cut end.

Further steps of connecting the vessel to the vascular port of the bionic organ are as in the ex vivo method according to the invention. Once the mandrel is removed, the opening in the vessel is closed with a vascular suture.
isolated arterial and venous vessels of the recipient in situ-here, steps a) to c) of the method according to the invention are replaced as follows: terminal vessels of the recipient (e.g. epigastric vein and artery, internal iliac vein and artery) isolated over a distance of at least 40 mm are temporarily closed, preferably with a vascular clamp, and an incision is made therein in the middle. An incision is then made in the vessel, preferably in the vicinity of both clamps, so as to introduce the systems according to the invention through the openings obtained towards the cut-off ends. The further steps of connecting the vessels to the vascular ports of the bionic organ (which, here, has vascular ports arranged at opposite ends) are as for the ex vivo method according to the invention. Once the mandrels are removed, the openings in the vessels are closed with a vascular suture.

The system for connecting a bionic organ to a vascular graft has undergone various modifications with respect to a commercially available stent in order to allow for it to be used in the method according to the invention.

The stent described in the present application is housed in a casing that keeps it in a compressed (retracted) state until it is removed from the casing, and it comprises a mandrel to allow it to be removed from the casing. Once the stent is removed from the casing, it expands and assumes the expanded state.

Such a stent may further comprise a depth indicator on its casing to allow for accurate positioning thereof during insertion, in particular during insertion into the vascular system of the bionic organ. Said depth indicator allows for precision embedding of the stent with the vascular graft in the inlet/outlet opening (vascular port) of the bionic organ so as to optimise insertion.

The stent described in this application may be provided with loops provided at each end thereof that extend beyond the casing of the stent. Said loops are used to fix the stent to the vascular graft using surgical thread. Relative to commercially available systems, the system disclosed in the present application is characterised by a reduced length of the casing and mandrel in the assembled state. Said length ranges from 20 to 40 cm, preferably equals 30 cm. The length thus reduced facilitates handling of the stent under laminar chamber conditions.

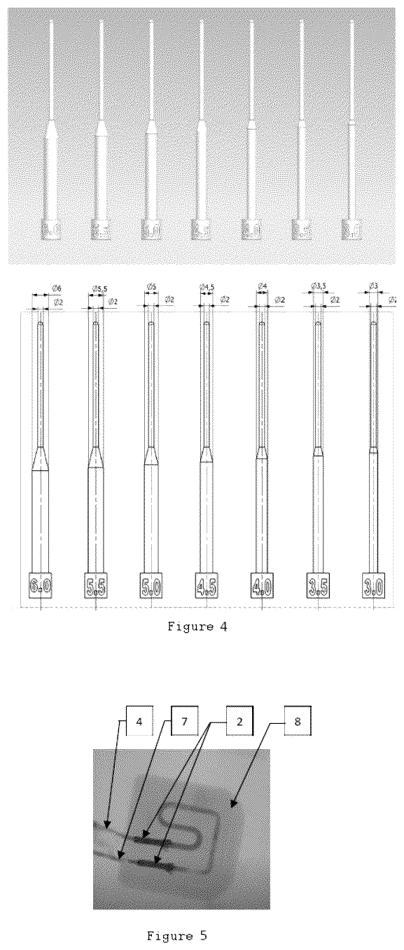

The stent is also available in a variety of diameter ranges and lengths of the stent itself. The diameters of the stent when fully expanded, wherein full expansion is meant to be the maximum expansion of the stent outside the vascular graft and the vascular port, are in the range of 0.2 to 50 mm, preferably 1 to 20 mm, and are suitably selected so that the diameter of the stent prior to expansion is less than that of the vascular graft and that the stent expands to a diameter greater than or equal to that of the vascular port. Preferably, the diameter of the stent after expanding is between 100% and 150% of the diameter of the vascular port. Examples of stents with various diameters are shown in FIG. 4. The length of the stent, in turn, ranges from 10 to 40 mm, preferably equals 20 mm. Such a range of available stent diameters and lengths provides extensive adaptation possibilities depending on a type of organ to be implanted and the diameter of the vascular graft and vascular port.

Furthermore, the system according to the present invention comprises a mandrel with a breakable tip. Said mandrel may be provided with a notch along its circumference at the level of the outer end of the stent, which allows the tip of the mandrel to break off in the determined place. The tip of the mandrel being breakable prevents damage to the bionic organ or vascular port during the insertion of the vascular graft thereto together with the stent. This solution also eliminates a need for the vessels in bionic organs to be designed in straight line with respect to the vascular ports. Breaking off of the tip prevents the mandrel from abutting against the vessel wall of the bionic organ when the stent is being pushed out, so that it does not press on the vessel wall.

The modifications of existing solutions as above make the system according to the invention a unique and currently unavailable product, that may play a crucial part in the field of transplantation, in particular in cases of transplantation of bionic organs.

Preferably, the system according to the invention comprises a set of samplers that allow to accurately tailor the diameter of the stent to the vascular graft. Such a set of samplers may include the following samplers with diameters from 0.2 to 50 mm, preferably from 1 to 20 mm, wherein the set preferably comprises samplers in the full range of diameters from 1 to 20 mm with 0.5 mm increments.

### EMBODIMENTS

### Example 1—Preparation of a Stent for Use in the Method According to the Invention Based on a Commercial Stent

In order to obtain a stent (2) suitable for use in the method according to the invention, the following modifications have been made relative to a commercially available stent with size of 6 mm×20 mm:
Shortening of the mandrel (1) and casing (3) set of the stent (2) to a length of 30 cm so that it can be handled ex vivo in a laminar chamber. For comparison, the length of commercial mandrel-casing sets is, for example, 125 cm;
Making a notch on the casing (3) of the stent (2) with a length of approximately 5 mm to allow subsequent placement of a surgical thread on the first loop of the stent without opening the stent;
Creating two fixing loops (6) on the outer edge of the stent (2), one at each end of the stent (2);
Creating a circumferential notch on the mandrel (1) in the proximity of the point where it retracts in the casing (3), so as to facilitate its subsequent breaking or twisting;
Creating a depth indicator (5) on the casing (3) of the stent (2).

### Example 2—Implementation of the Connection Between Vascular Graft and Artificial Pancreas (Ex Vivo Stage of Artificial Pancreas Transplantation)

### 1) Creation of a Vascular Graft

Pig splenic arteries obtained from a local abattoir were used to create the graft (4). Using gauges, vessels with a diameter of 3.5 to 4 mm and a length of approximately 60 mm were selected. The vessels were then subjected to flow decellularization in a closed system at a constant flow rate of 40 mL/min. The decellularization process consisted of the following steps:
48 h flow of the solution of 1% Triton X-100+0.1% NH4OH in 1×PBS at a temperature of 4° C.,
48 h flow of 1×PBS solution with 0.01% streptomycin at a temperature of 4° C.,
8 h flow of 1×PBS solution with 0.0002% DNase I and 0.12 mM Ca²⁺ and Mg²⁺ at a temperature of 37° C.,
48 h flow of 1×PBS solution with 0.01% streptomycin at a temperature of 4° C.

The vessels were then preserved in 1×PBS solution with 0.01% streptomycin and subjected to radiation sterilisation at a radiation dose of 25 kGy. Finally, the vessels were repopulated with recipient's cells (using recipient's endothelial cells).

### 2) Stent Selection

A self-expanding stent (2) with a diameter of 6 mm and a length of 20 mm, obtained as described in example 1, was used in the experiment.

### 3) Artificial Pancreas to be Connected to a Vascular Graft

A bio-printed organ, an artificial pancreas (8) was used in the experiment. The organ (8) was obtained by means of extrusion bioprinting technology using a bioink, which is the subject of another patent application EP19218191.5 by the same applicants, which is still pending at the date of filing this application. Organ housing components (8) were printed using SLA technology with photo-curable polymers. Inlet and outlet-vascular ports (7) of the artificial pancreas (8) had identical diameters of 4 mm.

### 4) Course of Experiment

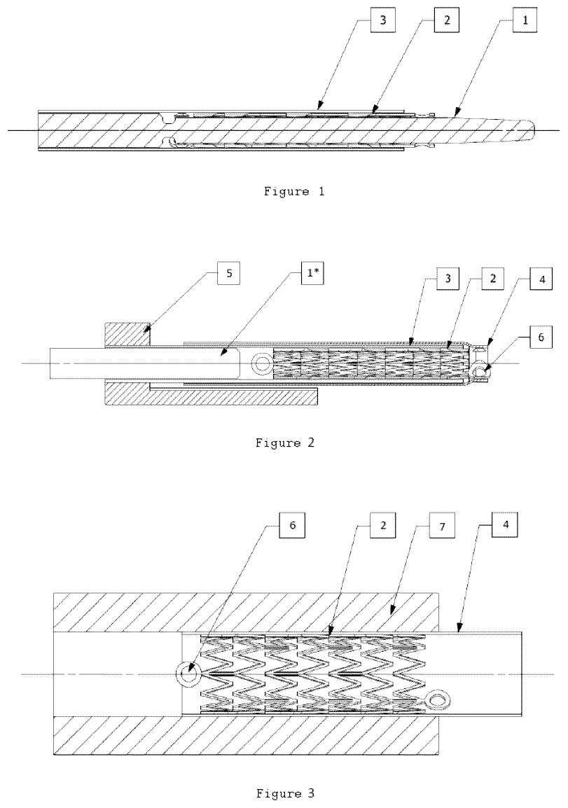

The method is schematically illustrated in FIG. 1-3. FIG. 1 shows the stent (2) in its compressed state prior to its inserting in the vascular graft (4). The stent (2) was in a compressed configuration and was situated inside the casing (3). The casing was equipped with a mandrel (1), which allowed simple and non-damaging insertion of the stent (2) into the vascular graft (4) thanks to its rounded, tapered tip.

In the first step of connecting the bionic organ (8) to the vascular graft (4), shown in FIG. 2, a stent (2) in a compressed configuration arranged in the casing (3) was inserted into the vascular graft (4) to the appropriate depth. A stent in the casing (3) was inserted into the vascular graft so that it was entirely located inside the graft.

Once the stent (2) together with the casing (3) was inserted into the vascular graft (4), the stent (2) was gently removed from the casing (3) so that it did not open completely to allow for fixing it to the vascular graft (4) via fixing loops (6) at both ends of the stent (2). This was achieved by suturing a loop (6) to the vascular graft (4) with surgical thread with the purpose to immobilise the stent (2) in the graft (4). The stent (2) was sutured to the vascular graft (4) using a single vascular suture (Prolen 4-0) at both ends of the stent (2). The tip of the mandrel (1) protruding beyond an outline of the casing (3) at a suitable, circumferentially notched point, was broken off. In this step, the tip has already fulfilled its function, i.e. it allowed for the vascular graft (4) to slide over the casing (3) and, more than being no longer necessary, it even hindered the execution of subsequent steps of the method. Two such structures connecting the vascular graft (4) to the stent (2) were prepared.

In the next step, shown in FIG. 3, a vascular graft (4) with a stent (2) sutured thereto in a casing (3) was inserted into the vascular port (7). The prepared structures connecting the vascular graft (4) to the stent (2) were inserted into the arterial and venous vascular ports (7) of the artificial pancreas (8), respectively, to a depth of 20 mm. This depth also corresponded to the length of the stent (2) used. The latter value was particularly important as it guaranteed proper fixing without undue enlargement of the entire organ. The inner diameter of the vascular graft (4) was selected such that it was 0.0-0.5 mm larger than the inner diameter of the vascular port (7). In this step, the insertion depth was controlled using a depth indicator (5).

In the next step, respectively, each of the two self-expandable stents (2) was removed from the casing (3) using a mandrel (1*), which caused the stent (2) to expand to an expanded state.

The casing (3) and the mandrel (1*) of the stent (2) were then removed from both vascular ports (7) of the artificial pancreas (8), which resulted in the stent (2) expanding into the expanded state, i.e. to a diameter corresponding to that of the inlet and outlet of the artificial pancreas (8), i.e. 4 mm, which in turn resulted in the expansion of the vascular graft (4) and immobilisation of the stents (2) together with the vascular grafts (4) in the lumen of the respective vascular ports (7). In this step, the connecting means was the expansion force of the stent (2), which ensured not only connection, but also fixation of the vascular grafts (4) in the vascular ports (7), while preventing stenosis along the length of the stent (2). Opening the stent (2) resulted in expansion of the proximal section of the stent (2) slightly (over a length of approximately 2 mm) beyond the contour of the vascular graft (4) being implanted. The exposed part of the stent (2) situated outside the graft (4) anchored the artificial pancreas (8) in the vascular port (7), and the further funnel-like tapering part sealed the vascular port (7).

### 5) Results

The artificial pancreas (8) obtained in this experiment is illustrated in FIG. 5. The figure shows the flow of a contrast medium through the pancreas (8), from the inlet vascular graft to the outlet vascular graft. Fixing of vascular grafts (4) with a stent (2) is marked black.

Ex vivo tests performed demonstrated the tightness of the connection at a pressure up to 180 mmHg. Above this value, tightness deficits were observed in the artificial pancreas (8), but they only involved a dissection in the functional part of the bionic pancreas (8) rather than the vascular ports (7).

The artificial pancreas (8) obtained as described in example 2, connected to the vascular grafts (4), was implanted into the body of a live pig by making an incision in the respective pig iliac blood vessels and connecting them using end-to-side Carrel sutures to the vascular graft (4) located in venous and arterial vascular ports (7) of the artificial pancreas (8), respectively. After 14 days, the artificial pancreas (8) was again removed and dissected for visual inspection.

The results of the in vivo experiment confirmed that the connection between the vascular graft (4) and the artificial organ (8) thus obtained was durable and tight.

1. A system for connecting a bionic organ to a vascular graft, characterised in that it comprises a self-expanding stent (2), a casing (3) and a mandrel (1) with a breakable tip, wherein in an assembled state of the system the stent (2) in a compressed state is placed in the casing (3), which casing (3) holds the stent (2) in a compressed state until the stent (2) is removed from the casing (3), and a mandrel (1) is placed inside the stent (2) for removing the stent (2) from the casing (3), wherein, in the assembled state, the length of the casing (3) and the mandrel (1) is in a range of 20 to 40 cm, the length of the stent (2) is in a range of 10 to 40 mm, and a diameter of the stent (2) is selected such that, in the compressed state, the diameter of the stent (2) is less than diameter of the vascular graft (4) and, when expanded, the diameter of the stent
(2) is greater than or equal to diameter of the vascular port (7) in the bionic organ (8).

2. The system according to claim 1 characterised in that in the expanded state, the diameter of the stent (2) ranges from 0.2 mm to 50 mm, preferably from 1 to 20 mm.

3. The system according to claim 1, characterised in that in the assembled state, the casing (3) and the mandrel (1) have a length of 30 cm, and the stent (2) has a length of 20 mm.

4. The system according to claim 1, characterised in that the mandrel (1) comprises a circumferential notch which allows the breakable tip of the mandrel (1) to be broken off.

5. The system according to claim 1, characterised in that the breakable tip of the mandrel (1) has a conical shape with a rounded end.

6. The system according to claim 1, characterised in that the stent (2) comprises at least one fixing loop (6), preferably two fixing loops, one at each end of the stent (2).

7. The system according to claim 1, characterised in that the casing (3) comprises a depth indicator (5).

8. The system according to claim 1, characterised in that it comprises a set of at least two samplers that allows for selecting the diameter of the stent (2) to match the diameter of the vascular port (7), wherein the samplers have diameters of different sizes ranging from 0.2 mm to 50 mm, preferably from 1 mm to 20 mm.

9. A method for connecting a bionic organ to a vascular graft under ex vivo conditions, characterised in that the connection is achieved by means of a system as defined in claim 1, wherein said method comprises the following steps:
a) Inserting the system in the assembled state into the vascular graft (4)
b) Partially removing the stent (2) from the casing (3) and attaching the stent (2) to the vascular graft (4), preferably by means of a loop (6)
c) Breaking off the tip of the mandrel (1), preferably at the circumferentially notched point d) Inserting the vascular graft (4) with the attached stent (2) in the casing (3) into the vascular port (7) of the bionic organ (8)
e) Removing the stent (2) from the casing (3) using the mandrel (1*)
f) Removing the casing (3) and the mandrel (1*) from the vascular port (7) of the bionic organ (8).

10. The method according to claim 9, characterised in that the following are used as the vascular graft (4):
a decellularized or recellularized vessel of animal origin, or
a preserved vessel of animal origin, or
an autogenous vessel, preferably a saphenous vein, or
a vessel harvested from a deceased donor and preserved, or
a vessel harvested from a deceased donor and non-preserved, or
a vascular prosthesis, preferably made of EPTFE (expanded polytetrafluoroethylene)

11. The method according to claim 9, characterised in that prior to implementing step a), the vascular graft (4) is decellularized and then populated with endothelial cells.

12. The method according to claim 9, characterised in that the bionic organ (8) is prepared using 3D bioprinting technology.

13. The method according to claim 9, characterised in that insertion depth in step d) is controlled by the depth indicator (5) and it corresponds to the length of the stent (2) used.

14. The method according to claim 13, characterised in that in step d) the stent (2) is inserted to a depth of 10 to 40 mm, more preferably 15 to 30 mm, and most preferably to a depth of 20 mm.

15. The method according to claim 9, characterised in that the sequence of steps a) to f) is performed for each of the vascular ports (7) of the bionic organ (8).
